(19) United States
(12) Patent Application Publication
(10) Pub. No.: US 2016/0199641 A1
(43) Pub. Date: Jul. 14, 2016

(54) DEVICE AND METHOD FOR NEURAL COCHLEA STIMULATION

(71) Applicant: ADVANCED BIONICS AG, Stäfa (CH)

(72) Inventor: Iris Arweiler, Hannover (DE)

(21) Appl. No.: 14/909,974

(22) PCT Filed: Aug. 19, 2013
(86) PCT No.: PCT/EP2013/067221

§ 371 Date: Feb. 3, 2016

(51) Int. Cl. A61N 1/36; H04R 25/00

(52) U.S. Cl. CPC A61N 1/36032; H04R 25/554; H04R 25/606; H04R 2225/021

(57) ABSTRACT

There is provided a device for neural stimulation of a patient's cochlea, comprising means for providing an input audio signal; a sound processor for generating, from the input audio signal, a neural stimulation signal for neural stimulation of the ipsilateral ear according to the hearing loss of the ipsilateral ear and a bone conduction stimulation signal for vibrational stimulation of the contralateral ear according to the hearing loss of the contralateral ear; an implantable cochlear implant stimulation arrangement comprising a plurality of stimulation channels for stimulating the cochlea of the ipsilateral ear according to the neural stimulation signal; and a bone conduction vibrator for stimulating the contralateral ear according to the bone conduction stimulation.

The invention relates to a device and method for neural stimulation of a patient's cochlea and a programming unit for adjusting the stimulation device.

The sense of hearing in human beings involves the use of hair cells in the cochlea that convert or transduce acoustic signals into auditory nerve impulses. Hearing loss, which may be due to many different causes, is generally of two types: conductive and sensorineural. Conductive hearing loss occurs when the normal mechanical pathways for sound to reach the hair cells in the cochlea are impeded. These sound pathways may be impeded, for example, by damage to the auditory ossicles. Conductive hearing loss may often be overcome through the use of conventional hearing aids that amplify sound so that acoustic signals can reach the hair cells within the cochlea. Some types of conductive hearing loss may also be treated by surgical procedures.

Sensorineural hearing loss, on the other hand, is caused by the absence or destruction of the hair cells in the cochlea which are needed to transduce acoustic signals into auditory nerve impulses. People who suffer from sensorineural hearing loss may be unable to derive significant benefit from conventional hearing aid systems, no matter how loud the acoustic stimulus is. This is because the mechanism for transducing sound energy into auditory nerve impulses has been damaged. Thus, in the absence of properly functioning hair cells, auditory nerve impulses cannot be generated directly from sounds.

To overcome sensorineural hearing loss, numerous auditory prosthesis systems (e.g., cochlear implant (CI) systems) have been developed. Auditory prosthesis systems bypass the hair cells in the cochlea by presenting electrical stimulation directly to the auditory nerve fibers. Direct stimulation of the auditory nerve fibers leads to the perception of sound in the brain and at least partial restoration of hearing function.

To facilitate direct stimulation of the auditory nerve fibers, a lead having an array of electrodes disposed thereon may be implanted in the cochlea of a patient. The electrodes form a number of stimulation channels through which electrical stimulation pulses may be applied directly to auditory nerves within the cochlea. An audio signal may then be presented to the patient by translating the audio signal into a number of electrical stimulation pulses and applying the stimulation pulses directly to the auditory nerve within the cochlea via one or more of the electrodes.

Typically, the audio signal, which usually is captured by a microphone, is divided into a plurality of analysis channels, each containing a frequency domain signal representative of a distinct frequency portion of the audio signal, wherein the frequency domain signal in each analysis channel may undergo signal processing, such as by applying channel-specific gain to the signals. The processed frequency domain signals are used for generating certain stimulation parameters according to which the stimulation signals in each stimulation channel is generated. The analysis channels are linked to the stimulation channels via channel mapping, The number of stimulation channels may correspond to the number of analysis channels, or there may be more stimulation channels than analysis channels, or there may be more analysis channels than stimulation channels. Various stimulation strategies are used, such as current steering stimulation (in order to stimulate a stimulation site located in between areas associated with two or more electrodes) and N-of-M stimulation (wherein stimulation current is only applied to N of M total stimulation channels during a particular stimulation frame).

An example for such a CI system with electrical cochlea stimulation is described in WO 2011/032021 A1.

Typically, neural stimulation of the cochlea occurs by electric pulses applied via an electrode array implanted within the cochlea; alternatively or in addition neural stimulation of the cochlea may occur via light pulses or heat pulses applied within the cochlea.

In general, cochlea implants can be used unilaterally or bilaterally, depending on the nature and the degree of the hearing loss on each of the two ears. However, the nature of the hearing loss may be different for the two ears, so that a cochlea implant may be indicated for only one of the ears, while the hearing loss on the other ear nevertheless should be treated. Such treatment of the non-implanted ear may involve the use of a conventional hearing aid or a bone conduction hearing aid.

US 2010/0145135 A1 relates to an implantable hearing prosthesis comprising an implantable bone conduction transducer which is provided with an audio signal via a transcutaneous link from an external signal processor.

WO 2008/143573 A1 relates to a bone conduction hearing aid comprising an implantable piezoelectric bone conduction transducer which receives the audio signal via a transcutaneous cable from an external signal processor.

WO 2007/024657 A2 relates to a bone conduction hearing aid comprising an external unit including a vibrator output transducer and a magnet for being fixed at the patient's head via magnetic coupling to an implanted fixation magnet.

US 2006/0233409 A1 relates to a hybrid hearing aid comprising, BTE-(behind-the-ear)-type sound processing device, a CI electrode array for electrical stimulation of the cochlea and a bone conduction output transducer for vibrational hearing stimulation. The BTE-type device has an electrical output to an induction coil and a bone conduction output. The induction coil may be integrated within the BTE housing. Low frequency stimulation may occur via the cochlear implant, while higher frequencies may be stimulated via sound waves.

US 2012/0109006 A1 relates to the fitting of a hybrid hearing prosthesis which may comprise a bone conduction hearing aid and a cochlear implant.

US 2013/0064404 A1 relates to a hearing prosthesis including a cochlea implant and/or a bone conduction device.

It is an object of the invention to provide for a neural cochlea device and method, which is relatively simple and compact and which nevertheless provides for an appropriate treatment of patients having an inner ear damage requiring electrical stimulation of the cochlea on one ear and a conductive/combined hearing loss on the other ear.

According to the invention, these objects are achieved by a device as defined in claim 1 and a method as defined in claim 12, respectively.

The invention is beneficial in that, by providing a common sound processor for a cochlear implant stimulation arrangement for stimulation of the ipsilateral ear and a bone conduction vibrator for vibrational stimulation of the contralateral ear, the need for an additional device, such as an electroacoustic hearing aid, for treatment of the contralateral ear or the need for additional surgery for bone anchoring of a hearing aid at the contralateral ear can be eliminated. The invention is particularly beneficial for patients having an indication for a cochlear implant only for the ipsilateral ear, while having a contralateral conductive hearing loss, a contralateral combined conductive and sensorineural hearing loss or a contralateral hearing loss which cannot be treated with a conventional hearing aid (for example due to a malformation of the contralateral ear, chronic inflammation of the contralateral ear canal, etc.).

Typically, the device comprises a headpiece to be fixed at the side of the ipsilateral ear for establishing a wireless transcutaneous link to the CI stimulation arrangement.

According to one embodiment, the bone conduction vibrator forms part of the headpiece.

According to an alternative embodiment, the bone conduction vibrator is implantable, typically into the mastoid, together with an implantable coil arrangement which may be used both to supply the neural stimulation signal to the cochlea implant stimulation arrangement and to supply the bone conduction stimulation signal to the bone conduction vibrator.

The sound processor preferably is part of a BTE unit worn behind the patient's ipsilateral ear,

Further preferred embodiments are defined in the dependent claims.

Hereinafter, examples of the invention will be illustrated by reference to the attached drawings, wherein:

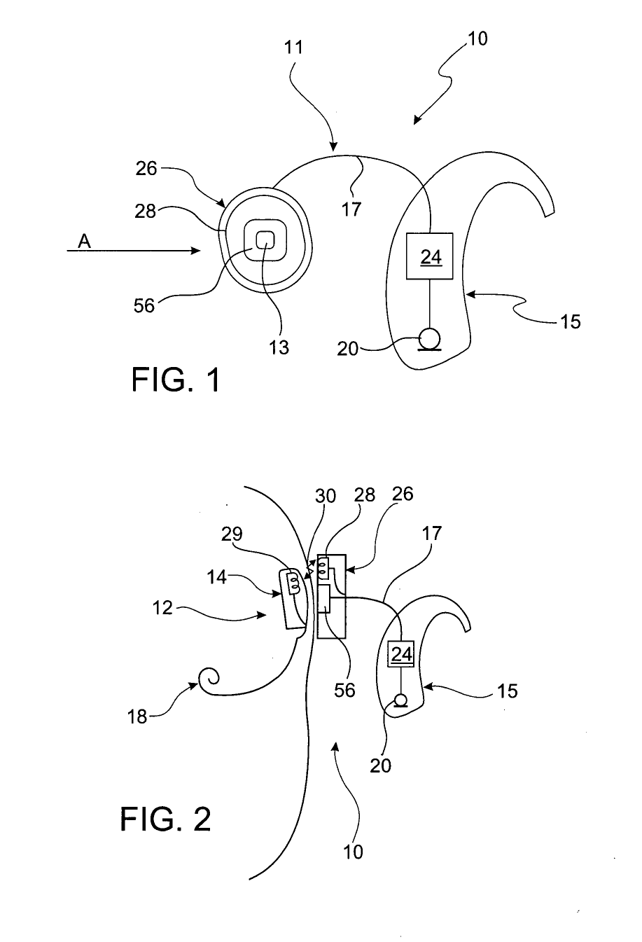

FIG. 1 is a schematic side view of an example of a hybrid hearing stimulation device according to the invention;

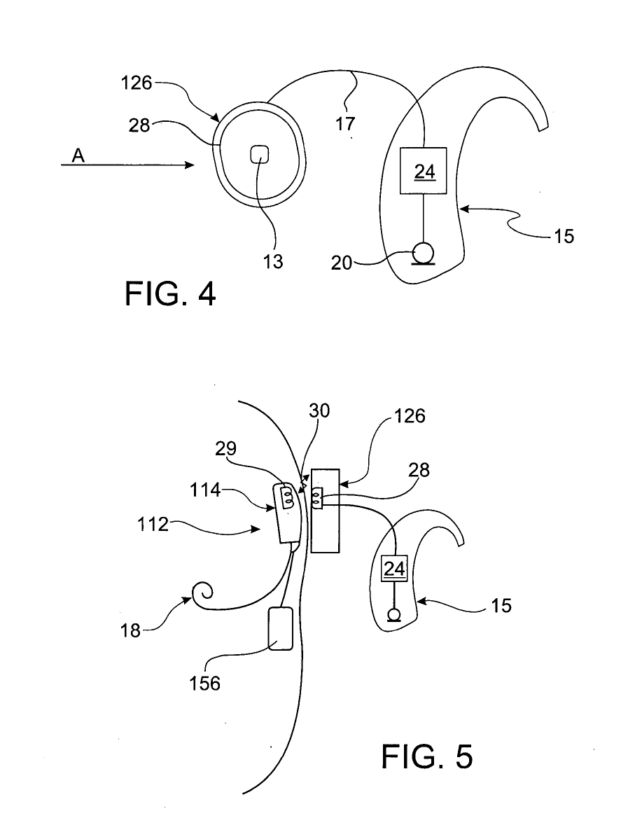

FIG. 2 is a view of the device of FIG. 1 seen in the direction of the arrow A in FIG. 1;

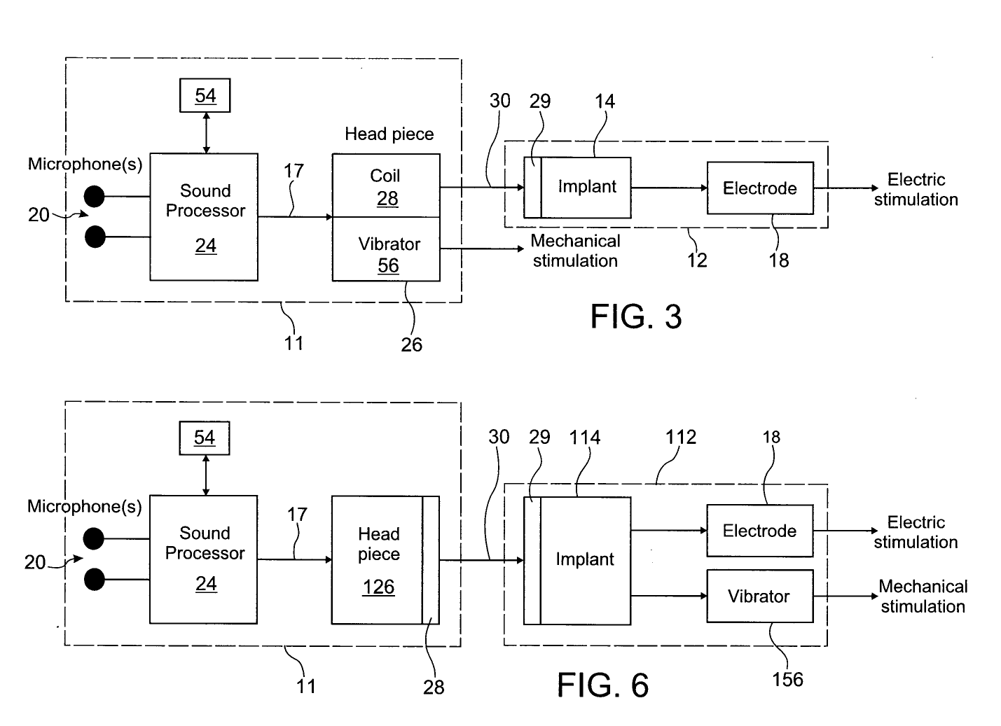

FIG. 3 is a block diagram of the signal processing of the device of FIG. 1;

FIGS. 4 to 6 are views like FIGS. 1 to 3, respectively, wherein, however, an alternative embodiment is shown;

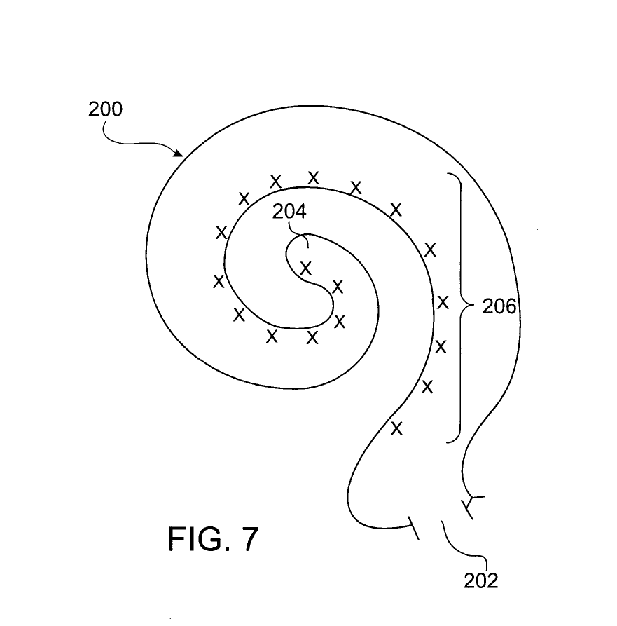

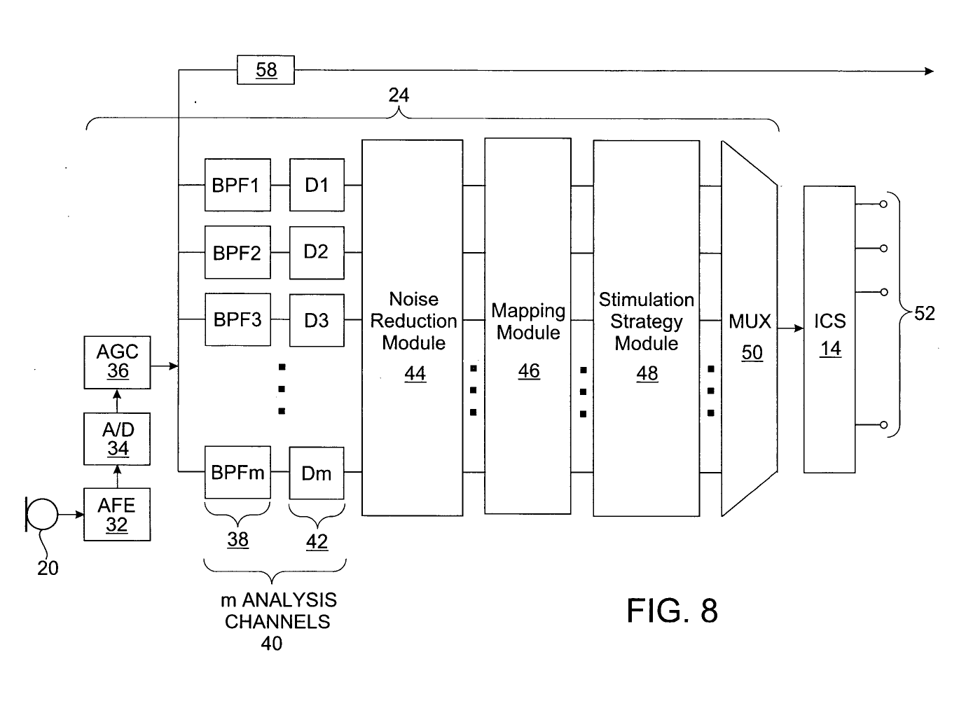

FIG. 7 is a schematic cross-sectional view of a human cochlea with marked stimulation sites; and

FIG. 8 is a more detailed block diagram of an example of the signal processing structure of a hybrid device according to the invention.

FIGS. 1 to 3 provide for a schematic illustration of an example of a neural stimulation system 10, comprising a sound processing subsystem 11 and an implantable stimulation subsystem 12, wherein FIG. 1 is side view which shows only the external part of the system 10, and wherein FIG. 2 is view seen in the direction of the arrow “A” of FIG. 1 which shows both the external part and the implantable part 12.

The sound processing sub-system 11 serves to detect or sense an audio signal and divide the audio signal into a plurality of analysis channels, each containing a frequency domain signal (or simply “signal”) representative of a distinct frequency portion of the audio signal. A signal level value and a noise level value may be determined for each analysis channel by analyzing the respective frequency domain signal, and a noise reduction gain parameter may be determined for each analysis channel as a function of the signal level value and the noise level value of the respective analysis channel. Noise reduction may be applied to the frequency domain signal according to the noise reduction gain parameters to generate a noise reduced frequency domain signal. Stimulation parameters are generated based on the noise reduced frequency domain signal and are transmitted to the stimulation sub-system 12.

Stimulation sub-system 12 serves to generate and apply electrical stimulation (also referred to herein as “stimulation current” and/or “stimulation pulses”) to stimulation sites at the auditory nerve within the cochlear of a patient in accordance with the stimulation parameters received from the sound processing sub-system 11. Electrical stimulation is provided to the patient via a CI stimulation assembly 18 comprising a plurality of stimulation channels, wherein various known stimulation strategies, such as current steering stimulation or N-of-M stimulation, may be utilized.

As used herein, a “current steering stimulation strategy” is one in which weighted stimulation current is applied concurrently to two or more electrodes by an implantable cochlear stimulator in order to stimulate a stimulation site located in between areas associated with the two or more electrodes and thereby create a perception of a frequency in between the frequencies associated with the two or more electrodes, compensate for one or more disabled electrodes, and/or generate a target pitch that is outside a range of pitches associated with an array of electrodes.

As used herein, an “N-of-M stimulation strategy” is one in which stimulation current is only applied to N of M total stimulation channels during a particular stimulation frame, where N is less than M. An N-of-M stimulation strategy may be used to prevent irrelevant information contained within an audio signal from being presented to a CI user, achieve higher stimulation rates, minimize electrode interaction, and/or for any other reason as may serve a particular application.

The stimulation parameters may control various parameters of the electrical stimulation applied to a stimulation site including, but not limited to, frequency, pulse width, amplitude, waveform (e.g., square or sinusoidal), electrode polarity (i.e., anode-cathode assignment), location (i.e., which electrode pair or electrode group receives the stimulation current), burst pattern (e.g., burst on time and burst off time), duty cycle or burst repeat interval, spectral tilt, ramp-on time, and ramp-off time of the stimulation current that is applied to the stimulation site.

FIG. 8 illustrates a schematic structure of the human cochlea 200. As shown in FIG. 8, the cochlea 200 is in the shape of a spiral beginning at a base 202 and ending at an apex 204.

Within the cochlea 200 resides auditory nerve tissue 206 which is organized within the cochlea 200 in a tonotopic manner. Low frequencies are encoded at the apex 204 of the cochlea 200 while high frequencies are encoded at the base 202. Hence, each location along the length of the cochlea 200 corresponds to a different perceived frequency. Stimulation subsystem 12 is configured to apply stimulation to different locations within the cochlea 200 (e.g., different locations along the auditory nerve tissue 206) to provide a sensation of hearing.

Returning to FIGS. 1 to 3, sound processing subsystem 11 and stimulation subsystem 12 are configured to operate in accordance with one or more control parameters. These control parameters may be configured to specify one or more stimulation parameters, operating parameters, and/or any other parameter as may serve a particular application. Exemplary control parameters include, but are not limited to, most comfortable current levels (“M levels”), threshold current levels (“T levels”), dynamic range parameters, channel acoustic gain parameters, front and backend dynamic range parameters, current steering parameters, amplitude values, pulse rate values, pulse width values, polarity values, filter characteristics, and/or any other control parameter as may serve a particular application.

In the example shown in FIGS. 1 to 3, the stimulation sub-system 12 comprises an implantable cochlear. stimulator (“ICS”) 14 and a stimulation assembly 18 comprising a plurality of stimulation contacts for electrical stimulation of the auditory nerve. The stimulation assembly 18 may be inserted within a duct of the cochlea in such a manner that the stimulation contacts are in communication with one or more stimulation sites within the cochlea, i.e. the stimulation contacts are adjacent to, in the general vicinity of, in close proximity to, directly next to, or directly on the respective stimulation site.

In the example shown in FIGS. 1 to 3, the sound processing sub-system 11 comprises a BTE unit 15 including a microphone arrangement 20 which comprises at least one microphone for capturing audio signals from ambient sound and a sound processor 24 which receives audio signals from the microphone arrangement 20 and a headpiece 26 having a coil 28 disposed therein. The sound processor 24 is configured to process the captured audio signals in accordance with a selected sound processing strategy to generate appropriate stimulation parameters for controlling the ICS 14 and may include, or be implemented within, a behind-the-ear (BTE) unit or a portable speech processor (“PSP”). In the example of FIGS. 1 to 3 the sound processor 24 is configured to transcutaneously transmit data (in particular data representative of one or more stimulation parameters) to the ICS 14 via a wireless transcutaneous communication link 30. The headpiece 26 may be affixed to the patient's head via magnetic forces between a magnet 13 of the headpiece 26 and an implanted magnet (not shown) and is positioned such that the coil 28 is communicatively coupled to the corresponding coil 29 included within the ICS 14 in order to establish the link 30. The link 30 may include a bidirectional communication link and/or one or more dedicated unidirectional communication links. According to an alternative embodiment, the sound processor 24 and the ICS 14 may be directly connected by wires.

In FIG. 8 a schematic example of a sound processor 24 is shown. The audio signals captured by the microphone 20 are amplified in an audio front end circuitry 32, with the amplified audio signal being converted to a digital signal by an analog-to-digital converter 34. The resulting digital signal is then subjected to automatic gain control using a suitable automatic gain control (AGC) unit 36.

After appropriate automatic gain control, the digital signal is subjected to a filterbank 38 comprising a plurality of filters F1 . . . Fm (for example, band-pass filters) which are configured to divide the digital signal into m analysis channels 40, each containing a signal representative of a distinct frequency portion of the audio signal sensed by the microphone 20. For example, such frequency filtering may be implemented by applying a Discrete Fourier Transform to the audio signal and then distribute the resulting frequency bins across the analysis channels 40.

The signals within each analysis channel 40 are input into an envelope detector 42 in order to determine the amount of energy contained within each of the signals within the analysis channels 40 and to estimate the noise within each channel. After envelope detection the signals within the analysis channels 40 may be input into a noise reduction module 44, wherein the signals are treated in a manner so as to reduce noise in the signal in order to enhance, for example, the intelligibility of speech by the patient. Examples of the noise reduction module 44 are described in WO 2011/032021 A1.

The noise reduced signals are supplied to a mapping module 46 which serves to map the signals in the analysis channels 40 to the stimulation channels S1 . . . Sn. For example, signal levels of the noise reduced signals may be mapped to amplitude values used to define the electrical stimulation pulses that are applied to the patient by the ICS 14 via M stimulation channels 52. For example, each of the m stimulation channels 52 may be associated to one of the stimulation contacts or to a group of the stimulation contacts.

The sound processor 24 further comprises a stimulation strategy module 48 which serves to generate one or more stimulation parameters based on the noise reduced signals and in accordance with a certain stimulation strategy (which may be selected from a plurality of stimulation strategies). For example, stimulation strategy module 48 may generate stimulation parameters which direct the ICS 14 to generate and concurrently apply weighted stimulation current via a plurality 52 of the stimulation channels S1 . . . Sn in order to effectuate a current steering stimulation strategy. Additionally or alternatively the stimulation strategy module 48 may be configured to generate stimulation parameters which direct the ICS 14 to apply electrical stimulation via only a subset N of the stimulation channels 52 in order to effectuate an N-of-M stimulation strategy.

The sound processor 24 also comprises a multiplexer 50 which serves to serialize the stimulation parameters generated by the stimulation strategy module 48 so that they can be transmitted to the ICS 14 via the communication link 30, i.e. via the coil 28.

The sound processor 24 may operate in accordance with at least one control parameter. Such control parameters may be the most comfortable listening current levels (MCL), also referred to as “M levels”, threshold current levels (also referred to as “T levels”), dynamic range parameters, channel acoustic gain parameters, front and back end dynamic range parameters, current steering parameters, amplitude values, pulse rate values, pulse width values, polarity values and/or filter characteristics. Examples of such auditory prosthesis devices, as described so far, can be found, for example, in WO 2011/032021 A1.

According to the invention, the neural stimulation device 10 also comprises a bone conduction output transducer or vibrator 56, with the sound processor 24 providing not only for a neural stimulation signal but also for a bone conduction stimulation signal to be supplied to the vibrator. According to the invention, the bone conduction stimulation signal is generated by the sound processor 24 according to the (measured) hearing loss of the contralateral ear, whereas the neural stimulation signal is generated according to the hearing loss of the ipsilateral ear. To this end, data corresponding to the measured hearing loss of the contralateral ear may be stored in a memory 54 connected to the sound processor 24. The bone conduction vibrator 56 is provided for vibrational/acoustic stimulation of the contralateral ear. Since bone conduction signals may travel through the patient's skull, the vibrator 56 preferably is located at the ipsilateral car, rather than at the contralateral ear, in order to realize a relatively compact and simple device.

According to the example of FIGS. 1 to 3, the vibrator 56 is provided as part of the head piece 26, acting on the patient's skull from outside the head.

The sound picked up by the microphone arrangement 20 is converted to an electrical signal (audio signal) and is amplified and frequency-shaped according to the measured hearing loss of the contralateral ear in the sound processor 24, with the processed audio signal being supplied to the headpiece 26, i.e. to the vibrator 56, via the electrical connection 17. The vibrator 56 converts the audio signal to mechanical vibrator energy which drives the vibrator 56. The vibration induced to the headpiece 26 results in a corresponding vibration of the skull, with the vibration propagating through the skull to the contralateral ear, where the vibration is picked up by the inner ear.

In the example of FIG. 8, a unit 58 is indicated schematically which provides for the amplification and frequency shaping of the audio signal to be supplied to the vibrator 56.

An alternative embodiment is shown in FIGS. 4 to 6, wherein the bone conduction vibrator 156 is implantable and forms a part of an implantable unit 112 of the device 110, rather than forming part of the headpiece 126. In this case, the vibrator 156 preferably is implanted into the mastoid, with the implantable coil 29 and the ICS 114 as usual being implanted in the temporal bone. The link 30 established by the coils 28 and 29 is used for supplying also the bone conduction stimulation signal provided by the sound processor 24 to the ICS 114 and thus to the vibrator 156.

In principle, the sound processor, rather than forming part of a BTE unit, could form part of a body-worn unit, which may be worn, for example, around the neck.

According to a further variant, the microphone for capturing ambient sound may be an implantable microphone rather than forming part of the BTE unit.

1. A device for neural stimulation of a patient's cochlea, comprising
means for providing an input audio signal;
a sound processor for generating, from the input audio signal, a neural stimulation signal for neural stimulation of the ipsilateral ear according to the hearing loss of the ipsilateral ear and a bone conduction stimulation signal for vibrational stimulation of the contralateral ear according to the hearing loss of the contralateral ear;
an implantable cochlear implant stimulation arrangement comprising a plurality of stimulation channels for stimulating the cochlea of the ipsilateral ear according to the neural stimulation signal; and
a bone conduction vibrator for stimulating the contralateral ear according to the bone conduction stimulation.

2. The device of claim 1, further comprising a headpiece to be fixed at the side of the ispsilateral ear at the head of the patient, the headpiece comprising an external coil arrangement for establishing a wireless transcutaneous link with the cochlear implant stimulation arrangement via an implantable coil arrangement in order to provide the cochlear implant stimulation arrangement with the neural stimulation signal.

3. The device of claim 2, wherein the headpiece is adapted to be fixed at the head via a magnetic arrangement.

4. The device of claim 2, wherein the bone conduction vibrator forms part of the headpiece.

5. The device of claim 2, wherein the bone conduction vibrator is implantable.

6. The device of claim 5, wherein the bone conduction vibrator, the cochlear implant stimulation arrangement and the implantable coil arrangement form part of an implantable unit, with the bone conduction vibrator being connected to the implantable coil arrangement for being supplied with the bone conduction stimulation signal via the transcutaneous link.

7. The device of claim 6, wherein the bone conduction vibrator is to be implanted into the mastoid.

8. The device of claim 1, wherein the sound processor is part of a BTE unit to be worn behind the patient's ipsilateral ear.

9. The device of claim 8, wherein the means for providing an input audio signal form part of the BTE unit.

10. The device of claim 1, wherein the means for providing an input audio signal comprise at least one microphone.

11. The device of claim 1, wherein the cochlear implant stimulation arrangement comprises a plurality of electrodes for electrical stimulation of the cochlea.

12. A method for neural stimulation of a patient's cochlea, comprising
providing an input audio signal; generating, from the input audio signal, a neural stimulation signal for neural stimulation of the ipsilateral ear according to the hearing loss of the ipsilateral ear and a bone conduction stimulation signal for vibrational stimulation of the contralateral ear according to the hearing loss of the contralateral ear;
stimulating, by an implantable cochlear implant stimulation arrangement comprising a plurality of stimulation channels, the cochlea of the ipsilateral ear according to the neural stimulation signal; and
stimulating, by a bone conduction vibrator, the contralateral ear according to the bone conduction stimulation.
